(19) United States
(12) Patent Application Publication
(10) Pub. No.: US 2010/0016683 A1
(43) Pub. Date: Jan. 21, 2010

(54) SMART INSTALL

(75) Inventors: Johannes Hendrikus Maria Lemmers, Reek (NL); Nicoline Haisma, Veldhoven (NL); David La Hei, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/522,919

(22) PCT Filed: Jan. 10, 2008
(86) PCT No.: PCT/IB2008/050086

§ 371 Date: Jul. 13, 2009

Related U.S. Application Data
(60) Provisional application No. 60/885,639, filed on Jan. 19, 2007.

(51) Int. Cl. A61B 5/00; G06F 3/048; G06F 3/01

(52) U.S. Cl. 600/301; 715/764; 715/744

(57) ABSTRACT

When installing components of a remote patient health monitoring system (14′), a generic off-the-shelf set top box (STB) (12) and home gateway (30) are configured by inserting a configuration carrier (42) into each device. The configuration carrier (42) has stored thereon configuration routines and information provided by the remote health monitoring service provider. Configuration information includes, without being limited to, network IDs and channel information, hardware ID information, and the like. The home gateway (30) is connected to the STB (12) using WiFi network link, and to a central server (20) over an Ethernet link. The home gateway (30) is further coupled to one or more patient monitoring devices using a wireless link, such as Bluetooth.

The present application finds particular application in remote communication of healthcare information and monitoring of health status for a patient, particularly involving user-friendly installation of networking systems at a patient's location, such as a patient's residence, to facilitate connecting a user to a centralized server for service. However, it will be appreciated that the described technique(s) may also find application in other remote user setup systems, other health status monitoring techniques, or other communication techniques.

Existing remote health systems are typically expensive to install. For example, a home system includes an Internet or other communication gateway and a set top box (STB) that connects to a television or the like to provide an interface to a user or patient, the STB employs hardware to satisfy interfacing requirements, such as video decoding, video storage, Bluetooth interfacing, support of Java and sufficient performance characteristics to permit acceptable UI responses. The gateway and STB may be combined in a common housing. In one approach, a “deviceKey” and server IP address are installed at the factory and individually set for each STB. Storage of a multitude of deviceKeys at the STB is costly, as is coordinating an assigned STB with the patient(s) to whom it is assigned. Conventional configuration mechanisms lead to separate stocks per deployment, which is undesirable because it impedes deployment of “off-the-shelf” devices. Moreover, the tight coupling between users and devices complicates replacement of devices or re-use of devices at another user location, and typically requires additional installation by a professional installer.

In another approach, unconfigured hardware is supplied to the patient along with a configuration guide. Many elderly or infirm patients are not able to complete the configuration process. Often, lengthy phone calls are made to a help line, using large amounts of technical assistance time, which is costly. Sending a technician to make a house call is even more costly, but often necessary.

With conventional factory-configured systems, various steps would be performed at the factory, including providing the STB with an encryption key and IP-addresses of one or more servers. An encryption key and MAC address of the STB are stored in a table in a database at the server. A professional installer performs the following steps at the user's home: setup the physical connection to a GUI, and the connection between the STB and modem; contacts an operator to associate the STB with the user at installation time; and installs the measurement devices with a Settings menu in the GUI. A precondition of the installation is that the modem is already configured (e.g., has a DSL, cable, or other high-speed Internet or broadband account). The modem is configured such that it provides an IP-address to the STB using, for example, a dynamic host configuration protocol (DHCP) or the like. Under the foregoing architecture and installation scheme, a considerable portion of the total cost of the health monitoring system can be attributed to installation.

With regard to installation of conventional STB systems, the cable between the STB and a broadband modem may be undesirable when both devices are separated by a large distance and/or in separate rooms. When the distance cannot be bridged with a cable, further complications arise. Additionally, the STB is always fully active and consumes maximum power to handle incoming measurement data. Many of these deficiencies are typical for connected (e.g., to the Internet) systems that provide user-oriented services. The present application provides new and improved remote patient health monitoring systems and methods, which overcome the above-referenced problems and others.

Similar problems are encountered by technically unsavy users connecting modems or gateways for other purposes.

In accordance with one aspect, a system for installing and configuring a remote healthcare system, includes a set top box (STB), a home gateway having a first reader thereon, and a configuration carrier that stores configuration information provided by a healthcare service provider for configuring both the STB and the home gateway. The configuration carrier is inserted into the first reader of the home gateway and the configuration information is read and stored to persistent memory to configure the home gateway to establish an Internet link to a central server and to establish a wireless access point for the STB.

In accordance with another aspect, a method for installing and configuring a remote patient healthcare system in a patient's residence includes constructing a configuration carrier that stores configuration information for a set top box (STB) and a home gateway, and applying power to the home gateway and connecting the home gateway to the Internet. The method further includes inserting the configuration carrier into the home gateway and automatically configuring the home gateway to establish a communication link with a central server, and connecting the STB to a GUI and applying power to the STB and the GUI. The configuration carrier is configured by the healthcare service provider.

One advantage is that installation cost associated with the monitoring system is reduced.

Another advantage resides in mitigating a need for expensive preconfigured home gateways and/or STBs.

Another advantage resides in encapsulating setup of the WiFi communication link on the configuration carrier to mitigate user error during system installation.

Still further advantages of the subject innovation will be appreciated by those of ordinary skill in the art upon reading and understand the following detailed description.

The innovation may take form in various components and arrangements of components, and in various steps and arrangements of steps. The drawings are only for purposes of illustrating various aspects and are not to be construed as limiting the invention.

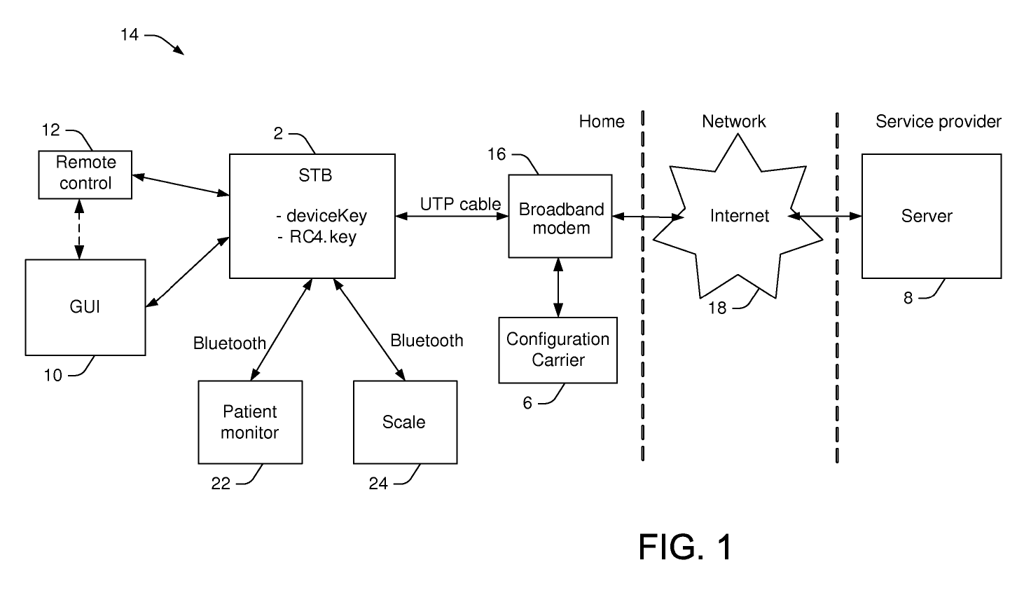

FIG. 1 is an overview of a remote health monitoring system for a patient, in accordance with one or more aspects set forth herein.

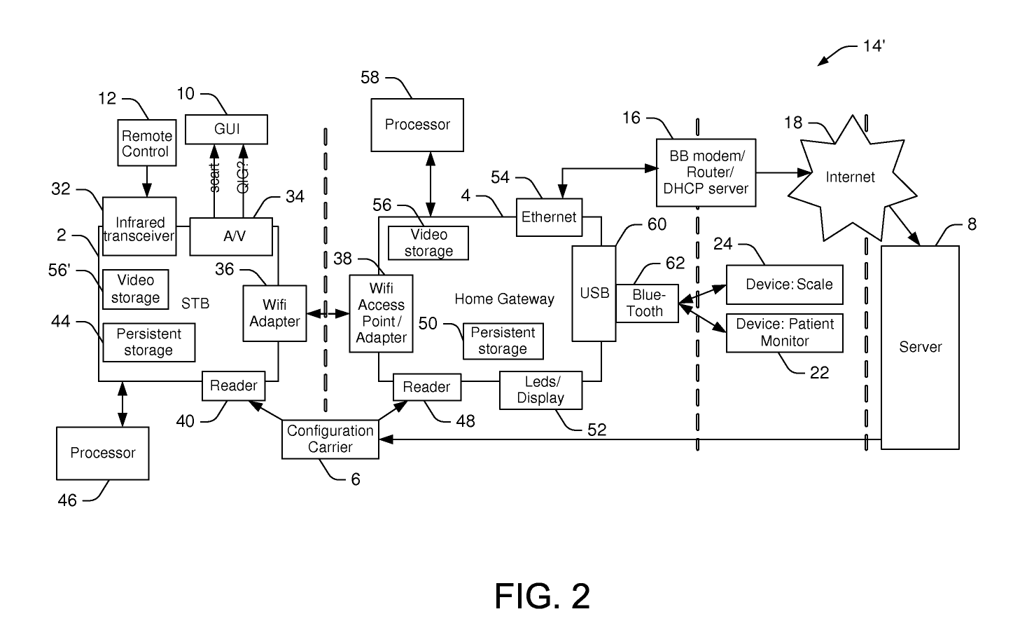

FIG. 2 depicts a remote health monitoring system for monitoring a patient in a remote location, such as the patient's home, in accordance with one or more aspects.

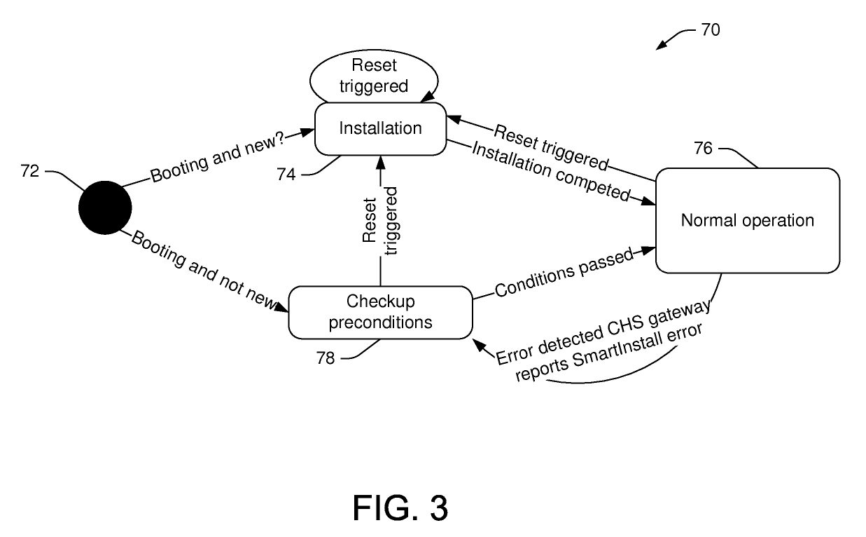

FIG. 3 is a state diagram depicting multiple states in both the STB and the gateway, in accordance with various aspects.

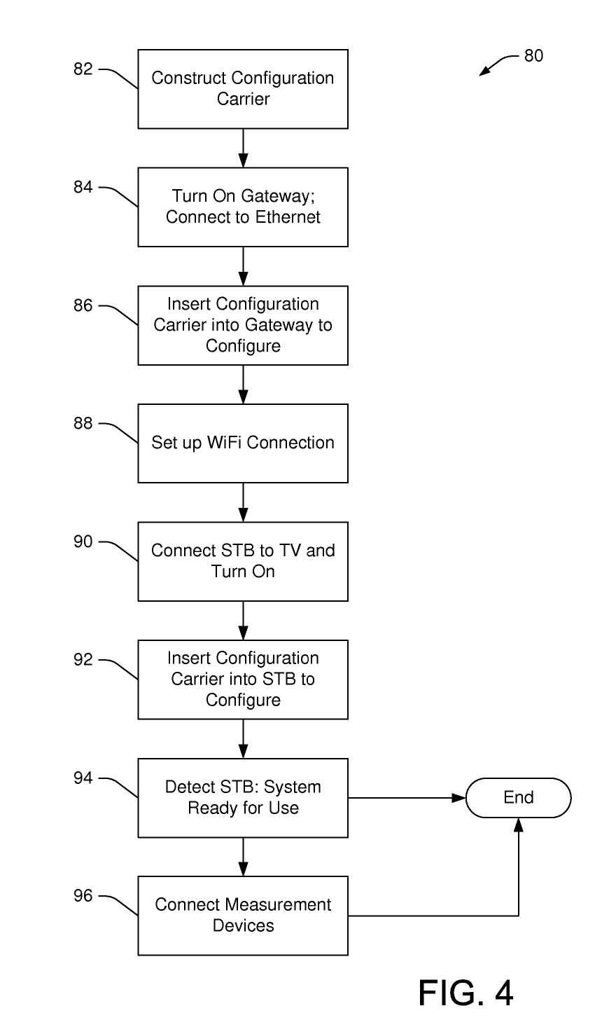

FIG. 4 illustrates a method for setting of an in-home health monitoring system for monitoring patient health status remotely at the patient's residence, in accordance with one or more aspects.

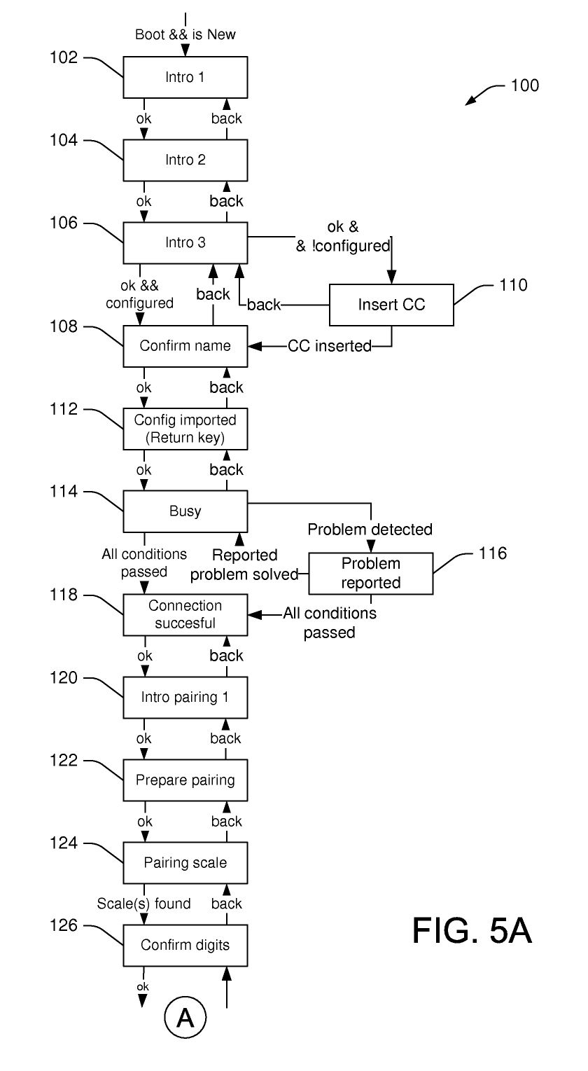

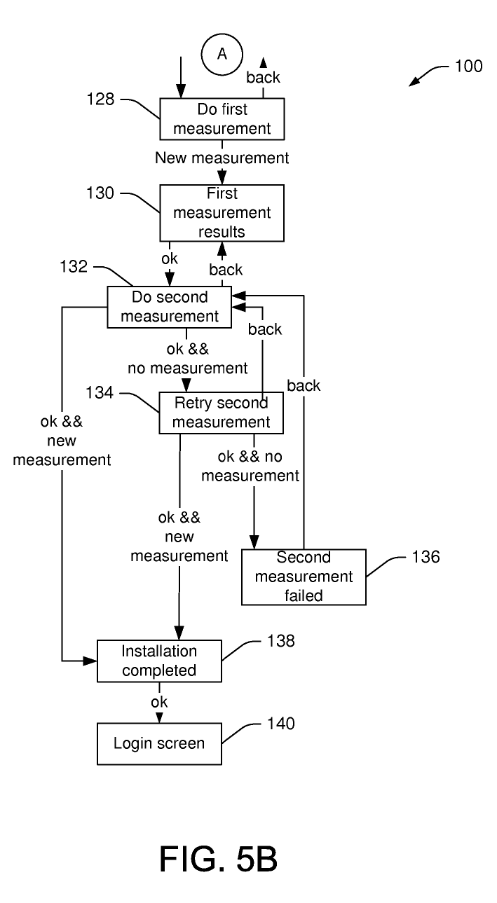

FIG. 5A illustrates a flow diagram of a method for smart installation of an STB.

FIG. 5B illustrates the continued flow diagram of the method of FIG. 5A.

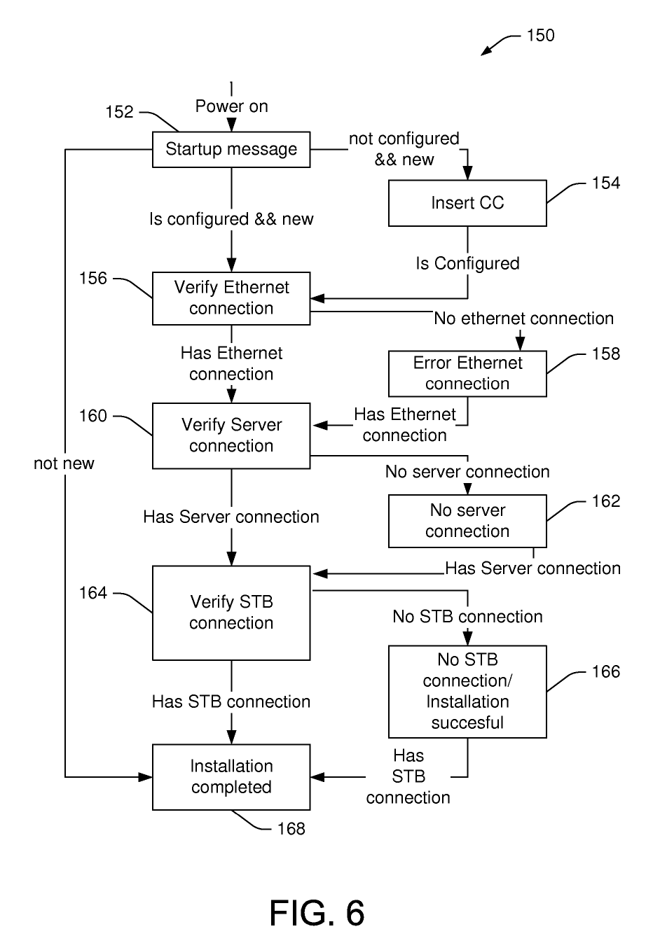

FIG. 6 illustrates a flow diagram of a method for smart installation of a home gateway component of a remote health monitoring system.

A remote patient health monitoring system includes a set top box (STB) and a WiFi gateway. It is desirable to configure the gateway to communicate with a server in an encrypted format. Similarly, the STB and the gateway need to be configured to communicate with each other in a secure and encrypted manner. Additionally, data packets may be further encrypted to limit access to patient data only to authorized personnel at the medical facility. Many elderly users of such a remote system are unfamiliar with configuring computer equipment, and configuration steps often prove to be beyond the ability of many potential users. One solution is to pre-configure the gateway and STB for the user at the factory. However, this approach is expensive and time-consuming, and is problematic when a component needs to be replaced or repaired. A second potential solution is to send a professional installer to install a generic gateway and STB and perform the configuration operations. This approach can add significant cost to the system. The present application proposes a simplified configuration arrangement to ensure that even an elderly, infirm adult can install and configure the remote health monitoring system.

According to one or more features, the patient receives or obtains an unconfigured STB 2 and an unconfigured gateway 4, along with a configuration carrier 6, such as a USB memory stick, smart card, bar-coded card, or the like. The patient connects the gateway unit 4 with the telephone line, cable line, or other source of Internet access, and connects the gateway unit 4 with a wall outlet or other source of power. Once the gateway unit 4 is powered up, the patient inserts the configuration carrier 6 into the gateway unit 4. The gateway unit 4 automatically reads the configuration information from the configuration carrier 6 and establishes a secure, encrypted communication link with a server 8.

In one aspect, the gateway 4 includes a communication interface for wireless (e.g. Bluetooth™, Zigbee™, or the like) communication with physiological monitoring devices such as a blood pressure cuff, a scale, and electrocardiogram device, an SpO₂ monitor, a manual patient input device, or the like. The gateway 4 stores these readings and communicates them to the server 8 (e.g., the gateway can regularly push data to the server, etc.).

In another aspect, the gateway device 4 communicates wirelessly with an STB 2 that is connected with a graphical user interface 10, such as the patient's television set, a computer monitor, or the like. In this aspect, the patient connects the output of the STB 2 with the input to the television set and connects the STB 2 with the wall outlet or other power supply. Then, the patient causes the STB 2 to read the configuration carrier 6 (e.g., by inserting the configuration carrier 6 into a port in the STB 2). With the information from the configuration carrier 6 and/or the gateway 4, the STB 2 completes a wireless, secure communication link with the gateway device 4 through which any additional configuration information can be shared. The STB 2 can be constructed with a significant memory to ensure that video clips and other information can be received over the Internet or other data link through the wireless device and stored in the STB 2 for patient viewing. A remote 12 for the television set can function as a manual physiological condition input device through which the patient can manually enter physiological parameters and other indicators of health and well-being, answer health-related questions, control the display of healthcare information, etc.

In another aspect, the functions of the STB 2, television set, and remote are integrated into a single unit including the STB 2 hardware and a touch screen monitor.

FIG. 1 is an overview of a remote health monitoring system 14 for a patient, in accordance with one or more aspects set forth herein. The system 14 includes a set top box (STB) 2 that is operatively coupled to a graphical user interface (GUI) 10 or other suitable device (e.g., a TV, laptop, PDA, etc.) through which a user or patient may interact (e.g., receive information from and enter information to) the STB 2. The STB 2 is further coupled to a broadband modem 16 (e.g., by an unshielded twisted pair (UTP) cable, coaxial cable, or the like), which facilitates accessing the Internet 18 or similar network to communicate with a server 8. A configuration carrier 6 can be inserted into a port on the modem to configure the modem 16, if desired, to configure the modem according to configuration information stored on the configuration carrier 6, in order to isolate an unsavy user from the configuration process. The configuration carrier 6 is configured by the service provider. Conventional systems, which do not employ a configuration carrier, require a costly professional installation. The STB 2 includes a device key and a security key, such as a Rivest Cipher 4 (RC4) or the like, and can be coupled to monitoring devices, including but not limited to a scale 22 that measures a patient's weight, and patient monitor 24 (e.g., to measure blood pressure, pulse rate, SpO₂, ECG, CO₂, or the like), etc. Additionally, a remote control 12 is provided for use by a patient to input information to the STB 2.

The STB 2 provides a user interface to the user or patient, which interface is shown on the GUI 10. Other functionality provided by the STB 2 includes: interfacing the monitoring devices, such as the scale 22 and the patient monitor 24 via Bluetooth and/or USB communication links; gathering measurements and forwarding measurement information to the server 8; identifying the user when communicating with the server 8, by retaining data; buffering user data and measurement data; etc. The user operates the STB 2 with the dedicated remote control 12, using infrared signals to input information, such as a key code. The patient monitor 24 and the scale 22 can be wired and/or wireless measurements devices, operate autonomously, and send the measurement data via Bluetooth or USB connection to the STB 2.

FIG. 2 shows another embodiment of remote health monitoring system 14′ for monitoring a patient in a remote location, such as the patient's home, in accordance with one or more aspects. Various features facilitate “smart” installation of the remote health monitoring system 14′ in a patient's home or other location remote from a centralized server. In this document, the smart install procedure and/or system is described with regard to with installing a remote health monitoring system at the home end (e.g., in a patient's home or the like). However, it will be appreciated that the smart install concept can also be applied to other similar systems, including but not limited to: wireless streaming data devices, multimedia hubs, future wellness gateways (e.g., fitness, weight management, sleep, etc), and the like.

An overview of the system 14′ is provided to facilitate understanding of the various components of the system 14′, which are described in greater detail below. The system 14′ includes a set-top box (STB) 2 that is operatively coupled to a home gateway component 4, which in turn is coupled to a broadband modem and/or router 16 to permit communication over the Internet 18 to a centralized server 8. The STB 2 includes an infrared or other wireless transceiver 32 through which a remote control 12 communicates with STB 2, as well as an audio/video (A/V) component 34 the provides information to a GUI 10 for presentation to the user. The GUI 10 and A/V component 34 can be connected by an SCART cable (e.g., a 21-pin connector, from the French acronym for Syndicat des Constructeurs d'Appareils Radiorécepteurs et Téléviseurs) or some other suitable connector (e.g., a coaxial cable, an N-pin connector, where N is an integer, etc.). The STB 2 also includes a WiFi adapter 36 that communicates with a WiFi access point or adapter 38 in the home gateway 4 to facilitate wireless bi-directional communication between the home gateway 4 and the STB 2. Still furthermore, the STB 2 includes a reader 40 that receives information from a configuration carrier 6. Configuration information includes, without being limited to, information associated with user identity, server identity, network identity, language preference, settings, monitoring device identity, encryption code protocol(s), etc. According to various examples, the reader 40 is a universal serial bus (USB) port and the configuration carrier 6 is a USB memory stick; additionally or alternatively, the reader 40 is a CD-ROM or DVD drive (or variant thereof) and the configuration carrier 6 is a CD or DVD that stores the configuration information. It will be appreciated that the reader 40 may be any suitable “reading” device capable for forming a connection with the configuration carrier 6, which may be any suitable portable storage device (e.g., a bar-coded card, a smart cart, a magnetic stripe card, a punch card, a key card, etc.), and receiving configuration information there from for configuring the STB 2, and that the foregoing examples are illustrative in nature and not intended to limit the scope of the above-described features.

The STB 2 additionally includes a persistent storage component 44 (e.g., a memory) that stores information associated with any and all functionality of the STB 2. For instance, configuration information received from the configuration carrier 42 via the reader 40 is stored in the persistent storage 44. A processor 46 is operatively associated with the STB 2 and executes instructions and/or routines stored in the persistent storage, such as one or more configuration routines, WiFi and/or other communication routines, etc. It will be appreciated that although the processor 46 is depicted as a separate component from the STB 2, the processor 46 may be included as a physical component in the STB 2.

The home gateway 4 includes a reader 48 that receives configuration information from the configuration carrier 6. The reader 48 may be similar or identical to the reader 40 of the STB 2, or may be substantially different from the reader 40, in which case an adapter can be employed to permit the reader 40 to read from the configuration carrier 6. Alternatively, the reader 48 may be designed to accept the configuration carrier 6 without an adapter, and the reader 40 can employ the adapter if the reader 40 is substantially different than the reader 48. In this manner, readers 40 and 48 permit a user to plug in the configuration carrier 6 to each of the STB 2 and the home gateway 4 to configure each device without requiring an expensive professional installation. The home gateway 4 also includes the WiFi access point/adapter 38 mentioned above, which communicates with the WiFi adapter 36 of the STB 2, as well as a persistent storage (e.g., memory) 50 that stores relevant information and/or routines for access and/or execution by a processor 58. Processor 58 and persistent storage 50 may be similar or identical to processor 46 and persistent storage 44, respectively, in structure, although information stored and/or executed by these components is not necessarily similar or identical.

The home gateway 4 additionally includes a display 52 that has one or more LEDs or other suitable indicator devices to indicate that the home gateway 4 is functioning properly. Moreover, the home gateway includes an Ethernet port 54 for communicating with the broadband modem/DHCP server 16, a video storage component 56 that stores video data, which may be transferred to the STB via the WiFi link for output to a user via the A/V component 34 and GUI 10. Additionally or alternatively, a video memory 56′ for performing all or some of these storage functions is located in the STB 2. Still furthermore, the home gateway 4 includes a USB port 60 that receives a Bluetooth component 62, which in turn communicates with one or more monitoring devices. For example, the patient monitor 22 and the scale 24 are depicted, although the system 14′ may include any number and/or type of monitoring devices, etc., as will be appreciated by those of skill.

In accordance with various features, the smart install mechanism uses the WiFi wireless networking protocol for communication between the GUI 10 and the home-end broadband access point 16. A device is therefore located at both sides of the WiFi connection: the STB 2 at the GUI 10 side, and the home gateway 4 at the broadband entrance point. The configuration carrier 6 is used to distribute the user-specific configuration from a healthcare service provider (not shown) to both the STB 2 and the home gateway 4. With the configuration information, the devices are able to connect to each other and to the central server 8. The service provider is able to dictate, via the configuration carrier 6, which network(s) and/or channel(s) the system 14′ will employ and therefore avoid (network or device) conflicts between neighboring users. It will be appreciated that alternatives to WiFi may be implemented in connection with various aspects, such as power line communication or the like.

A first part of the installation is performed by the user, prompted by a quick installation guide (QIG) that is provided by an audio/video (A/V) component 36 in the STB 2 to the GUI 10. If an issue arises during installation that is not addressed by the QIG, the user can call a helpdesk for detailed support. A second part of the installation is guided via the GUI 10. When the distance between the GUI 10 and the Internet entrance at the broadband modem 16 is large, bridging the gap with a network cable becomes problematic, and wireless communication (WiFi) becomes a practical alternative. The Smart Install concept fully shields configuration for the user by embedding the WiFi network in the system 14′ and by positioning a device at both ends of the WiFi connection. The configuration carrier 6 is used for exchange of the WiFi configuration, in order to ensure both devices can communicate with each other. The user is thus enabled to install a complex home end system without support of professional installers. Thus, by isolating potentially complicated WiFi link generation from the user, and by providing step-by-step instructions via the QIG presentation on the GUI 10, the user can quickly and inexpensively set up the system 14′.

The configuration carrier 6 is used to distribute the configuration data from a service provider to the home-end system (e.g., the STB 2 and gateway 4), and contains information to setup the connection from the STB 2 and gateway 4 to the server 8. Configuration information can include, without being limited to, encrypted extensible markup language (XML) data or the like, with specific fields for associated information (e.g., a user ID field, one or more hardware ID fields, etc.) According to other examples, configuration information can include network identification numbers (e.g., WiFi network ID numbers) network frequencies, patient profile information (e.g., user ID, health condition, language preference), software applications or portions thereof, etc., and any other suitable information for performing the described actions and configuring the system 14′. Configuration information can also include identification information, such as the patient's personal encryption code, languages, resources or software parts.

As a result, the STB 2 and gateway 4 do not need to be specifically preconfigured (e.g., by a manufacturer or the like) and are independent from service provider, country and language. It is therefore possible to manufacture a stock of generic devices instead of various stocks for specific service providers, countries and/or languages. Additionally, replacement of devices and re-use of devices is made much simpler. Moreover, the gateway 4 can distinguish between a measuring device intended for the user or patient and a measuring device intended for communication with a neighbor's system. For instance, a hardware identification number associated with the user's scale can be used to distinguish the user's scale from a scale employed in a neighbor's health monitoring system to mitigate confusion between measuring devices. Such as scenario can occur, for example, where multiple systems 14′ are employed in a nursing home, apartment building, or a nearby house. Additionally or alternatively, patient data can be linked (e.g., two patients can be linked to each other and to a single system or device), such as is described in U.S. application No. 60/755,0535, to permit sharing of devices. For instance, an elderly couple that requires remote healthcare or subscribe to the healthcare service can share a system 14′ and associated devices, and the system can distinguish between the patients while permitting each patient to use it.

According to some features, the configuration carrier 6 is a storage device, such as a USB memory stick or other portable memory device. A service provider writes the WiFi configuration, user identification information, and other configuration data to the configuration carrier. At installation time, the user inserts the configuration carrier into both the STB 2 and the home gateway 4. In this manner, both devices are configured such that they can connect to each other and to the remote service (e.g., via the server 20). Additionally, the configuration carrier 6 provides a mechanism to identify a specific user at the home-end. After the connection is established, the full configuration is exchanged. According to an example, the service provider writes (e.g., stores) the configuration data on the configuration carrier 6 and gives or sends it (for example by mail) to a new client. The configuration carrier has one or more graphical markings to explain to the user how to insert the configuration carrier 6. Moreover, the user's name can be printed on the configuration carrier 6 to mitigate confusion. When limited video storage is desired, the configuration carrier 6 can also be used as video storage, to save the cost of the hard disk and/or reduce bandwidth utilized to download the video material.

The configuration carrier 6 acts as a medium to distribute the configuration data, which enables use of “off the shelf” STB 2 devices, mitigates a need for special configuration in the factory, and permits utilization of the same STB model in different countries and for different services providers. Other advantages provided by the configuration carrier 6 include enabling reconfiguration of replacement devices, ease of explanation and execution, cost-effectiveness, quick connection of system devices, and increased security (e.g., because RC4 keys are distributed separately from hardware, which can be provided with an embedded mechanism to descramble scrambled keys). The content of the configuration carrier 6 can be encrypted to protect privacy sensitive data, the STB 2 and gateway 4 can be equipped with a decryption algorithm to access the configuration data.

According to still other features, peripheral measurement devices, (e.g., an electronic scale, a BP cuff, an SpO₂ monitor, a blood-glucose monitor, and the like) can be designed to be configurable by the configuration carrier 6. For example, the configuration carrier 6 can store configuration information for configuring a measurement device to communicate wirelessly with either or both of the home gateway 4 and the STB 2. In this example, a measurement device can be equipped with a reader for reading the configuration carrier and persistent memory for storing configuration information and the like.

As stated above, the configuration carrier 6 can be a USB memory stick, which is very cost-effective and is available from a variety of manufacturers. The user inserts the stick into a USB port, and data on the stick is freely accessed and decrypted. According to another example, the configuration carrier 6 is a near field communication (NFC) card, which provides a contactless (e.g., less than 10 cm) interface to store and retrieve data, which in turn permits usage of user-friendly constructions. The NFC mechanism permits control of access to the data.

According to another example, the configuration carrier 6 is a memory card. Memory cards are commonly used, for example, in digital cameras and high-end phones. According to this example, the readers 40 and 48 of the STB 2 and the gateway 4, respectively, are memory card readers. The data on the card is also freely accessed and decrypted. In yet another example, the configuration carrier 6 is a Smartcard. In yet another embodiment, the configuration carrier 6 includes a cell phone or PDA.

The configuration data stored on the configuration carrier 6 includes without being limited to: the host name and port number of the servers; the user's name, to be confirmed during installation; a card ID representing the user; RC4 keys to be used to encrypt a message (e.g., an initial message) to the server 8; a full WiFi configuration for the WiFi adapters 36, 38 to establish a successful connection between the home gateway 4 and the STB 2; etc.

The gateway 4 is responsible for interfacing with the various components in the system 14′ and interfacing to external systems. In this regard, the gateway 4 acts as gateway to the Internet and as interface to the measurement devices 22, 24. Additionally, the gateway 4 acts as WiFi access point to the STB 2 (and optionally other system components). According to other aspects, the gateway 4 includes a VoIP connection to connect a telephone (not shown).

The home gateway 4 and/or the STB 2 collects measurement data from the measurement devices 22, 24 and forwards the measurement data to the server 8. Additionally, the gateway 4 and/or the STB 2 acts as a data cache that caches information retrieved from the server 8 and/or caches measurement data until it is sent to the server 8. By connecting the measurement devices to the gateway 4 rather than the STB 2, the measurement devices can monitor patient status even when the STB 2 is off. The gateway 4 and/or the STB 2 can additionally pre-download video material to be presented to the user. The home gateway 4 communicates its (and/or the STB's) status to the user via LEDs and/or a display 52. The QIG information contains a reference to the LEDs combinations or display message and provides one or more actions that a user can perform in response to any problems, should one occur. The home gateway 4 provides the following advantages, including but not limited to enabling encapsulation of the WiFi network by the various devices, thus permitting installation the system 14′ with a minimum number of cables. By encapsulation the WiFi connection setup, the WiFi configuration is handled fully by the system 14′ and therefore protected from human error during setup. Another advantage is that the home gateway 4 can easily be installed to new and to existing DSL solutions, because it is based on Ethernet and DHCP. Moreover, the gateway 4 enables use of a standard STB 2 (for example a browser card) when the home gateway 4 is functioning as a web server. In this scenario, a user interface is available although no connection with the server 8 is available (e.g., during installation). If desired, the web server functionality can be split over the home gateway 4 and the central server 8.

According to other features, the gateway 4 facilitates decreasing standby power consumption, since the STB 2 can be placed in standby mode and/or switched off. Moreover, because the gateway 4 does not have to support a user interface and video decoding, low-cost hardware can be utilized in its manufacture. The gateway can also act as a data cache with the server 8 when expensive and/or low-bandwidth communication techniques are used, such as GPRS or UMTS. Still furthermore, the gateway 4 can be employed in other system configurations, with only measurement functionality and/or telephone support, if desired.

The STB 2 facilitates interaction with the user, and is located close to the GUI 10 and optionally operated with a remote control 12. The STB 2 has a WiFi adapter 36 to communicate wirelessly with the home gateway 4. The STB 2 is responsible for generating the user interface of the system 14′, which can be implemented as a stand-alone application or as a web-based user interface in a browser. In the latter case, the STB 2 becomes a very low-cost and straightforward web browser STB 2. This approach also enables the use of alternative user interface devices such as a streaming media device and/or a personal computer with a web browser. Further, when the video storage is in the gateway 4, the patient has the option of viewing the educational videos on any of a plurality of home televisions, computers, etc., that have an STB 2 or the equivalent configured to communicate with the gateway 4.

As stated above, the gateway 4 is coupled to one or multiple measurements devices, for example a scale 24 and/or a patient monitor 22. It is to be understood that although FIG. 2 depicts a scale 24 and a patient monitor 22 as measurement devices, the subject innovation is not limited to such. Rather any measurement device capable of measuring a condition or state of the patient can be coupled to the gateway 4 (e.g., via a wireless communication link). For instance, measurement devices can include without being limited to, a thermometer, a respiration monitor, a pulse rate monitor, a blood oxygen monitor, a blood pressure monitor, etc. Additionally, because the measurement devices are wirelessly coupled to the gateway 4, they can be located anywhere in the user's residence.

According to other aspects, the system 14′ can included fewer than all illustrated components. For instance, the system 14′ can employ one or more measurement devices without a GUI 10, if desired. According to some features, the user may interact with the system 14′ via a telephone (not shown), cellular phone, text messaging protocol, or the like. In other aspects, the system 14′ can include the GUI 10 without measurement devices. Still other configurations include the GUI 10, measurement devices, and a VoIP or telephonic connotation to the service provider.

Other alternative features relate to utilizing a laptop or tablet PC as the STB 2, in which case a mouse, keyboard, stylus, or other input device is utilized to enter information to the STB 2. In such a scenario, a computer screen serves as the GUI 10, and the computer thus combines the functionality of the STB 2, the GUI 10, the remote 12, and the various other components of the STB 2.

FIG. 3 is an illustration of a state diagram 70 depicting multiple states in both the STB 2 and the gateway 4, in accordance with various aspects. An initial condition 72 represents a starting state of the system (e.g., system 14′ or a variant thereof), wherein the system is not yet configured. From the initial condition 72, a determination is made regarding whether the system is a new system or whether the system is already configured (e.g., is not new). “New,” as used herein, can refer to a system or device that has never been initialized or configured as well as to a system that has been configured, installed, registered, etc., in the past but that has been restored to an uninstalled condition and therefore requires configuration and/or installation. Thus, “new” is not to be construed as including only devices that have never been used. If the system is new, then installation is performed at state 74. If the system is not new, then a checkup of various preconditions is performed at the checkup state 78. During the installation state 74 and the checkup conditions state 78, the devices verify various conditions required associated with creating a basic communication link between the STB 2, the home gateway 4 and the server 6. If a problem is detected, it can be reported to the user and QIG and/or GUI can provide a suggestion to solve the problem and/or a telephone number for a helpdesk that the user can call.

If all preconditions are satisfied during state 78, then a reset is triggered and the system proceeds to the installation state 74 for reinstallation, if necessary. Additionally or alternatively, the system proceeds to state 76 if all conditions are satisfied, where the system can proceed with normal operation (e.g., if no reinstallation is required). In the event that the gateway 4 detects an error, the error may be reported to the user (e.g., via the GUI 10 and/or the display 52), and the system returns to state 78 to determine whether a reset condition is present that triggers a reinstallation.

Once installation is completed, the system advances from the installation state 74 to the normal operation state 76. If a reset is triggered (e.g., due to a power failure or other event making reset desirable), then the system returns to the installation state 74 for reinstallation. Additionally, a reset can be triggered during the installation 74, which may be cased by an interruption of an installation protocol or the like.

FIGS. 4-6 illustrate one or more methods related to installing and/or configuring a remote health monitoring system for a patient, in accordance with various features. While the methods are described as a series of acts, it will be understood that not all acts may be required to achieve the described goals and/or outcomes, and that some acts may, in accordance with certain aspects, be performed in an order different that the specific orders described.

FIG. 4 illustrates a method 80 for setting of an in-home health monitoring system for monitoring patient health status remotely at the patient's residence, in accordance with one or more aspects. At 82, a configuration carrier is constructed. The configuration carrier can be a USB memory stick (e.g., a “jump drive”), or other portable memory device, including but not limited to, a NFC card, a memory card, a Smartcard, etc.). Configuration data is stored on the configuration carrier by a service provider, and includes, without being limited to user ID information associated with a particular user, or patient, with whom the configuration carrier is associated, as well as any and all configuration information needed for configuring an STB and a home gateway in the user's place of residence, or any other desired place, such as an office or the like. The configuration data includes information to facilitate setting up a WiFi connection between the STB and the gateway, as well as any other desired system setup information.

At 84, the gateway component is turned on and connected to the Internet (e.g., via an Ethernet connection or any other suitable wired/wireless connection). At 86, the user inserts the configuration carrier into the gateway, which reads the configuration data from the configuration carrier and proceeds to configure itself accordingly. Optionally, once the Internet connection is established, additional configuration and/or other setup data can be downloaded from a remote site. At 88, a WiFi adapter/access point associated with the gateway is configured according to the configuration information. The user connects the STB to a GUI, such as a television, at 90, and turns on the STB and GUI components. Once the STB and GUI are connected and have power, the user inserts the configuration carrier into the STB, at 92. The STB reads configuration data from the configuration carrier and configures itself accordingly. Once the STB is configured, the gateway can detect the presence of the STB, initiate a WiFi connection therewith, and the system is ready for use, at 94. If measurement devices (e.g., a scale, a BP cuff, a blood-oxygen monitor, etc.) are to be employed, they are connected to the gateway (e.g., via a wired or wireless connection, or both, a Bluetooth communication link, etc.), at 96. Optionally, the configuration carrier is inserted into an appropriate connector on each measurement device to configure it for secure (e.g., encrypted) communication with the gateway and/or STB.

FIG. 5A illustrates a flow diagram 100 of a method for smart installation of an STB, such as the STB 2 described above with regard to preceding figures. When the STB is powered on and is new (e.g., not currently configured and/or installed), a first introduction screen is presented to a user (e.g., via a GUI, such as a television), at 102. The user can click through or respond to the first introduction screen (e.g., using a remote control for the television, the STB, or another input device), and can navigate back and forth through a second introductory screen at 104, a third introductory screen at 106, etc., while the STB attempts to configure itself. If the STB is able to configure itself, then at 108, the user confirms his/her name or user ID.

If the STB is unable to configure itself, then at 110 a user is prompted to insert a configuration carrier containing information provided by a service provider to configure the STB. Once the configuration carrier is inserted, the method either reverts to 106 to re-present an introduction screen to the user to proceeds to 108 to prompt the user to confirm his/her name or user ID. Upon user confirmation, configuration information is imported to the STB from the configuration carrier, at 112. The configuration information can include a return key that enables the STB to decode and/or decrypt information from the configuration carrier as well as from a home gateway component. Optionally, at 114, a “busy” indicator is presented to the user to indicate to the user that the system is configuring itself. If a problem is detected, then at 116 the problem is reported. For instance, if the problem reported to the user, the user can consult the quick installation guide (QIG), which can be a printed manual or can be resident on the STB or the configuration carrier, in which case the user can navigate through the QIG using the remote control. Once all conditions are satisfied, the method proceeds to a “connection successful” state during which an indication of a successful connection between the STB and an associated home gateway component is presented to the user, at 118. At 120, an introduction “pairing” screen is presented to the user via the GUI, which indicates that a connection between the home gateway component and a measuring device is imminent. Although the measuring device described with regard to this example is a scale, it will be understood that the innovation is not limited to the scale as a measuring device, but rather may include any suitable or desired measuring device.

At 122, an indication is presented to the user that the pairing, or connection, between the gateway and the scale is preparing, and at 124 a “pairing” screen is presented to the user to indicate that an attempt to establish the connection is in progress. At 126, one or more digits representing the identity of the measurement device, or scale, are confirmed. Point “A” is labeled after act 126 to indicate a continuation point for the method, which is continued in FIG. 5B.

FIG. 5B illustrates the continued flow diagram 100 of the method described above, which continues from act 126 of FIG. 5A. At 128, a first measurement is taken. For instance, if the measurement device is a scale, as described according to this example, then the user is prompted to get on the scale at 128. The results of the first measurement are presented to the user at 130. At 132, the user is prompted to get on the scale again and a second measurement is taken. If a second measurement does not register, then the user is prompted to retry the second measurement at 134. From 134, the method can revert to 132 for a second measurement. Alternatively, if no new measurement is detected after a predetermined number of attempts, then at 136 a determination is made that the second measurement has failed. The method then reverts to 132 to prompt the user to retry the measurement. If a new measurement is detected (either in response to the prompt at 132 or the prompt at 134), then at 138 the user is provided with an indication that system installation is complete. The user is then presented with a login screen, at 140, where the user enters specific account information to log in to the monitoring system. Other embodiments need not employ measurement steps.

FIG. 6 illustrates a flow diagram 150 of a method for smart installation of a home gateway component of a remote health monitoring system. At 152, after power is applied to the gateway component, a user is presented with a startup message indicating that the gateway component is starting. If the gateway component is already configured and but not installed or registered, etc., then at 156 a verification is made regarding the presence of an Ethernet connection. For example, the verification can be internal to the gateway component, such that the gateway component checks to ensure that the connection is present, or the user can be prompted to connect an Ethernet cable to the gateway component, or both.

If the gateway component is new and not configured, then at 154 the user is prompted to insert a configuration carrier that has configuration information stored thereon, before the method proceeds to 156. If no Ethernet connection is detected at 156, then at 158, the user is presented with an indication of the connection error and prompted to correct the error by connecting an Ethernet cable to the gateway component. Once the Ethernet connection is detected, at 160, a server connection is verified to ensure that the gateway is in communication with a centralized server, such as at a hospital or other healthcare providing facility or the like. If no server connection is detected, then at 162 an indication of the server connection error is presented to the user, who then consults the QIG or calls a provided helpdesk number for further assistance. If the server connection is present, then at 164, a connection between the gateway and an STB is verified. If no connection there between is detected, then at 166 the user is informed of the error and prompted to connect and/or install the STB. Once the STB connection is verified, the user is informed that the installation of the home gateway component is successfully installed, at 168. In this manner, the user is taken through installation of the home gateway component step-by-step, and the configuration of a WiFi connection between the gateway and the STB is performed without user involvement, thereby reducing the risk of error during setup.

1. A system for installing and configuring a remote healthcare system (14′), including:
a set top box (STB) (2);
a home gateway (4) having a first reader (48) thereon; and
a configuration carrier (6) that stores configuration information provided by a healthcare service provider for configuring both the STB (2) and the home gateway (4);
wherein the configuration carrier (6) is inserted into the first reader (48) of the home gateway (4) and the configuration information is read and stored to persistent memory (50) to configure the home gateway (4) to establish an Internet link to a central server (8) and to establish at least one of a wireless or power line communication access point for the STB (2).

2. The system according to claim 1, wherein the STB (2) includes a second reader (40) into which the configuration carrier (6) is inserted, and which stores the configuration information to persistent memory (44) to configure the STB (2) and to establish at least one of a wireless or power line communication link with the home gateway (4).

3. The system according to claim 2, wherein the system (14′) monitors health status of a patient in a remote location.

4. The system according to claim 1, wherein the STB (2) is coupled to a graphical user interface (GUI) (10) that presents information to a patient.

5. The system according to claim 4, wherein the GUI (10) is a television set.

6. The system according to claim 4, wherein the patient enters information into the STB (2) using a remote control (12).

7. The system according to claim 4, wherein the configuration carrier additionally stores information associated with at least one of user profile, language preference, resources, or software, for configuring the GUI (10) and identifying the patient to the central server (8).

8. The system according to claim 1, further including at least one monitoring device coupled to one of the home gateway (4) and the STB (2) via a wireless communication link, wherein the at least one monitoring device monitors a physiological condition of the patient.

9. The system according to claim 8, wherein the at least one monitoring device is at least one of an electronic scale, a blood pressure (BP) monitor, a pulse rate monitor, and an SpO₂ monitor.

10. The system according to claim 8, wherein the at least one monitoring device is configurable by the configuration carrier to generate the wireless communication link.

11. The system according to claim 2, wherein the STB (2) is a personal computer having a built-in GUI (10), and wherein the patient employs at least one of a mouse and a keyboard to input information into the STB (2).

12. The system according to claim 2, wherein the configuration carrier (6) is at least one of a universal serial bus (USB) portable memory stick, a DVD, a CD-ROM, a smartcard, a key card, a bar-coded card, a near field communication card, or a magnetic strip card.

13. The system according to claim 2, wherein the configuration component (6) includes:
a routine (50) for configuring a wireless connection between the STB (2) and the home gateway (4);
a routine (48) for configuring the home gateway (4);
a routine (54) for configuring the STB (2);
a routine (56) for detecting the STB (2) at the home gateway (4); and
a routine (58) for connecting one or more measurement devices (22; 24) to the home gateway (4) using a wireless communication protocol.

14. A method for configuring the system of claim 1, comprising:
storing the configuration information in the configuration carrier (6);
inserting the configuration carrier (6) into the home gateway (4); and
with the configuration information from the configuration carrier (6), configuring the home gateway (4) to establish a communication link with the central server (8).

15. A method of installing and configuring a remote patient healthcare system (14′) in a patient's residence, including:
constructing a configuration carrier (6) that stores configuration information for a set top box (STB) (2) and a home gateway (4);
applying power to the home gateway (4) and connecting the home gateway (4) to the Internet;
inserting the configuration carrier (6) into the home gateway (4) and automatically configuring the home gateway (4) to establish a communication link with a central server (8);
connecting the STB (2) to a GUI (10) and applying power to the STB (2) and the GUI (10);
wherein the configuration carrier is configured by a healthcare service provider.

16. The method according to claim 15, further comprising automatically configuring a wireless access point at the home gateway (4) for the STB (2) upon insertion of the configuration carrier (6) into the home gateway (4), and inserting the configuration carrier (6) into the STB (2) and automatically configuring the STB (2) to establish a wireless communication link with the home gateway (4), or employing configuration information stored in persistent memory (44, 50) of one or both of the STB (2) and the home gateway (4) to automatically generate the wireless link.

17. The method according to claim 16, further including providing an indication to the patient, via the GUI (10), that the STB (2) has been detected by the home gateway (4) and the system (14′) is ready for use.

18. The method according to claim 16, further including connecting at least one measurement device (22, 24) to the home gateway (4), wherein the at least one measurement device measures a state of the patient and provides the measurement information to the home gateway (4) for transmission to the server (8).

19. The method according to claim 18, wherein the at least one measurement device includes at least one of a digital scale (24), a blood pressure monitor, a pulse rate monitor, a respiration monitor, and an SpO₂ monitor.

20. The method according to claim 15, wherein constructing the configuration carrier (6) further includes storing configuration instructions to the configuration carrier (6) before providing the configuration carrier (6) to the patient.

21. The method according to claim 20, wherein the configuration carrier (6) is at least one of a USB memory stick, a DVD, a CD-ROM, a smartcard, a key card, a bar-coded card, a near field communication card, or a magnetic strip card.

22. A processor (46, 58) or computer medium (6) programmed to perform the method of claim 15.

23. The method according to claim 15, further comprising storing information associated with at least one of user profile, language preference, or software to the configuration carrier and employing the stored information to configure the GUI (10) and to identify the user to the central server (8).

24. The method according to claim 15, further comprising employing the configuration carrier (6) to configure a replacement STB (2) or home gateway (4) upon malfunction of a previous device.

25. A home gateway (4) that facilitates providing healthcare service to a remote patient, including:
a reader (48) that receives a configuration carrier (6) having configuration information stored thereon;
a network port (54) that receives a network connection to permit communication between the home gateway (4) and a central server (8); and
a wireless adapter (62) that provides a wireless connection to one or more patient monitoring devices.

26. The home gateway (4) according to claim 25, further comprising a wireless adapter (38) that provides a wireless connection to a set top box (STB) (2).

27. A configuration carrier (6) for configuring a remote healthcare system, including:
a computer-readable medium having stored thereon computer-executable instructions for configuring one or more of an STB (2) and a home gateway (4) and for generating a communication link between the STB (2) and the home gateway, and an Internet link between the home gateway (4) and a central server (8);
wherein configuration information is stored to the configuration carrier by a healthcare service provider, and the configuration carrier is inserted into each of the home gateway (4) and the STB (2) by a patient in a remote location.

28. The configuration carrier (6) according to claim 27, further comprising information associated with at least one of user profile, language preference, resources, or software, for configuring a GUI (10) coupled to the STB (2) and for identifying the patient to the central server (8).

29. The configuration carrier (6) according to claim 27, wherein the computer-readable medium is at least one of a USB memory stick, a DVD, a CD-ROM, a smartcard, a key card, a bar-coded card, a near field communication card, and a magnetic strip card.

30. A system for installing and configuring a remote network, including:
a set top box (STB) (2);
a home gateway (4) having a first reader (48) thereon; and
a configuration carrier (6) that stores configuration information provided by a service provider for configuring both the STB (2) and the home gateway (4);
wherein the configuration carrier (6) is inserted into the first reader (48) of the home gateway (4) and the configuration information is read and stored to persistent memory (50) to configure the home gateway (4) to establish an Internet link to a central server (8) and to establish a wireless access point for the STB (2), and is inserted into a reader (40) of the STB (2) and the configuration information is read and stored to persistent memory (44) to configure the STB (2) to establish a wireless communication link with the home gateway (4).

31. The system according to claim 30, wherein the configuration carrier (6) further includes identification information specific to a user of the system, including at least one of user profile information or language preference.
